(19) United States
(12) Patent Application Publication
(10) Pub. No.: US 2008/0021552 A1
(43) Pub. Date: Jan. 24, 2008

(54) Apparatus To Facilitate Implantation

(76) Inventor: Shlomo Gabbay, New York, NY (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/830,020

(22) Filed: Jul. 30, 2007

Related U.S. Application Data
(63) Continuation-in-part of application No. 10/987,605, filed on Nov. 12, 2004.
Application No. 10/987,605 is a continuation-in-part of application No. 10/266,380, filed on Oct. 8, 2002.
Application No. 10/266,380 is a continuation-in-part of application No. 09/973,609, filed on Oct. 9, 2001.

(51) Int. Cl. A61F 2/02

(52) U.S. Cl. 623/11.11

(57) ABSTRACT

An apparatus includes a flange, a sleeve having a sidewall that extends longitudinally from the flange and terminates in a distal end portion, and a structure for substantially preventing flow of fluid in at least one axial direction from the distal end portion toward the flange. The structure can have a substantially closed condition and an open condition.

### RELATED APPLICATIONS

This application is a continuation-in-part of U.S. patent application Ser. No. 10/987,605, which was filed on Nov. 12, 2004, and entitled APPARATUS TO FACILITATE IMPLANTATION, which is a continuation-in-part of U.S. patent application Ser. No. 10/266,380, which was filed on Oct. 8, 2002, and entitled HEART VALVE PROSTHESIS AND SUTURELESS IMPLANTATION OF A HEART VALVE PROSTHESIS, which is a continuation-in-part of U.S. patent application Ser. No. 09/973,609, which was filed on Oct. 9, 2001, and entitled HEART VALVE PROSTHESIS AND SUTURELESS IMPLANTATION OF A HEART VALVE PROSTHESIS, the entire contents of each of the above-identified applications being incorporated herein by reference.

### TECHNICAL FIELD

The present invention relates to implantation of a prosthesis and, more particularly, to an apparatus and method to facilitate implantation.

### BACKGROUND

Various types of prostheses have been developed and corresponding approaches are utilized to implant prostheses in both human and non-human patients. It is well known to utilize annuloplastic rings, stents, heart valves and other implantable devices for helping improve cardiac operation valves in human patients. Oftentimes implantation of a prosthesis requires passage of the prosthesis through surrounding tissue, such as when the prosthesis is being implanted at a site located within an organ or within a protective covering or sheath (e.g., skin muscle). For example, to surgically implant a heart valve prosthesis into a patient, the patient typically is placed on cardiopulmonary bypass during a complicated, but common, open chest and usually open-heart procedure. Such procedures tend to be invasive to the patient and, in certain situations, may present great risk.

### SUMMARY

The present invention relates generally to an apparatus to facilitate implantation of an article, such as a prosthesis, an implant or other device.

One aspect of the present invention provides an introducer apparatus. The apparatus includes a flange, a sleeve having a sidewall that extends longitudinally from the flange and terminates in a distal end portion, and a structure for substantially preventing flow of fluid in at least one axial direction from the distal end portion toward the flange. The structure has a substantially closed condition and an open condition.

Another aspect of the present invention provides an implantation system. The system includes an implanter for discharging an article into a position within a patient, and an introducer apparatus for guiding the implanter to the position within the patient. The implanter includes an elongate member. The introducer apparatus includes a flange, a sleeve having a sidewall that extends longitudinally from the flange and terminates in a distal end portion, and a structure operatively associated with the sleeve. The structure is at least partially located within an interior of the introducer apparatus for substantially preventing flow of fluid in at least a first axial direction.

Yet another aspect of the present invention provides a system including means for discharging an article into a position within a patient, means for guiding the discharging means to the position within the patient, the guiding means including a flange, a sleeve having a sidewall that extends longitudinally from the flange and terminates in a distal end portion, means for controlling axial flow of fluid through the guiding means; and means for securing a handle member to the flange for facilitating insertion of the guiding means into a patient.

### BRIEF DESCRIPTION OF THE DRAWINGS

FIG. 1 depicts an example of an introducer apparatus in accordance with an aspect of the present invention.

FIG. 2 depicts an example of the introducer apparatus of FIG. 1 demonstrating operation thereof in conjunction with an elongate member in accordance with an aspect of the present invention.

FIG. 3 depicts an example of an implanter apparatus for implanting a prosthesis in accordance with an aspect of the present invention.

FIG. 4 depicts an example of an introducer apparatus being inserted at an in the aorta of a heart in accordance with an aspect of the present invention.

FIG. 5 depicts an example of a valve being implanted at an aortic position in accordance with an aspect of the present invention.

FIG. 6 depicts an example of an introducer apparatus being inserted near an apex of a heart in accordance with an aspect of the present invention.

FIG. 7 depicts an example of a valve being implanted at the pulmonic position in accordance with an aspect of the present invention.

FIG. 8 depicts an example of an introducer apparatus in accordance with another aspect of the present invention.

FIG. 9 depicts an example of an introducer apparatus in accordance with still another aspect of the present invention.

FIG. 10 depicts an example of an introducer apparatus in accordance with yet another aspect of the present invention.

FIG. 11 depicts an example of an introducer apparatus in accordance with still another aspect of the present invention.

FIG. 12 depicts an example of an introducer apparatus in accordance with yet another aspect of the present invention.

FIG. 13 depicts an example of an introducer apparatus being inserted into an aorta in accordance with an aspect of the present invention.

FIG. 14 depicts an example of another introducer apparatus being inserted near an apex of a heart in accordance with an aspect of the present invention.

FIG. 15 depicts an example of a valve being implanted at the aortic position in accordance with an aspect of the present invention.

FIG. 16 depicts an example of a valve being implanted at the pulmonic position in accordance with an aspect of the present invention.

### DESCRIPTION

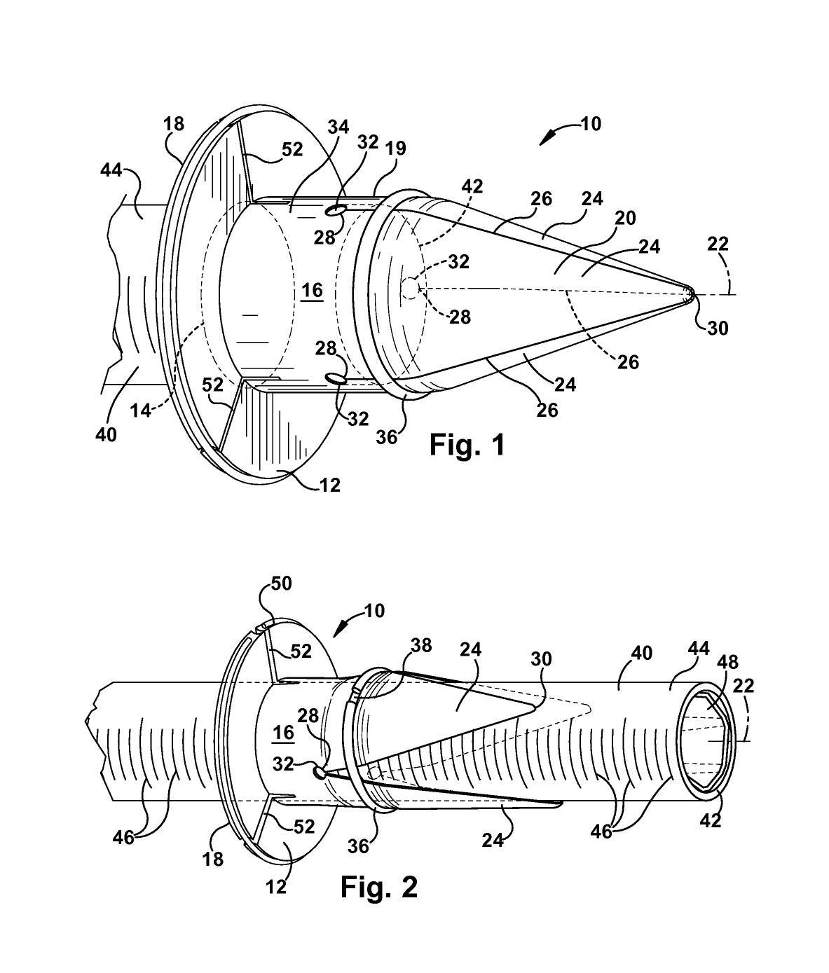

FIGS. 1 and 2 depict an example of an introducer apparatus 10 that can be implemented in accordance with an aspect of the present invention. The introducer apparatus 10 includes a flange 12. While the flange 12 is illustrated as a complete annular flange, those skilled in the art will understand and appreciate that the flange 12 can be implemented in a variety of shapes (e.g., rectangular hexagonal, etc.) and that flange 12 need not extend in a complete annular structure. For example, the flange 12 can be implemented as substantially co-planner tabs, a rim, as well as a curved or c-shaped flange portion or as a thicker sidewall portion of the apparatus 10.

In the example of FIG. 1, the flange 12 includes an inner periphery 14 that defines an opening into an associated sleeve 16. While the inner periphery is 14 generally circular in the examples of FIGS. 1 and 2, it can have other shapes. The flange 12 also includes an outer periphery 18 spaced apart from the inner periphery 14 by an associated intermediate portion thereof.

The sleeve 16 has a sidewall 19 that extends longitudinally from the flange 12 and terminates in a distal end portion 20. A central axis 22 extends through a center of the sleeve 16 and through the opening defined by the inner periphery 14 of the flange 12. An adjustable opening is operatively associated with the distal end portion. The adjustable opening includes means movable from at least a closed condition (FIG. 1) to an open condition (FIG. 2) to permit substantially free movement of an article, such as an implanter or trocar, axially through a passage defined by the sidewall 19.

By way of example, the adjustable opening in the distal end portion 20 can include two or more jaw members 24 that are movable in a generally radially direction relative to the axis 22 between the open and closed conditions. In the example of FIG. 1, the distal end portion 20 includes a plurality of three jaw members 24 positioned in closed condition. Longitudinally extending side edges of adjacent jaw members 24 define longitudinally extending slits 26. The longitudinally extending slits 26 extend through the sidewall 19 from a first position 28, which is located proximal to the flange 12, to intersect an opposed end 30 of the distal end portion 20 to separate the jaw members 24. An aperture 32 further may extend through the sidewall 19 of the sleeve at the first position 28 of each of the respective slits 26. The respective apertures 32 operate as hinges to facilitate the generally radial movement of the respective jaw members 24 from the closed condition to the open condition. Other structure, such as hinges, can also be employed to provide for moveability of the jaw members 24. Additionally, one of the slits might extend completely through the sidewall 19 as well as through the flange 12 to accommodate differently sized elongate members within the passage. Thus, the sidewall can be generally cylindrical (such as shown in FIGS. 1 and 2, or the sidewall can be generally C-shaped.

In the example of FIG. 1, the sleeve 16 can include an intermediate portion 34 located between the flange 12 and the distal end portion 20. The intermediate portion 34 of the sleeve 16 can have a generally right circular cylindrical cross section that extends a predetermined length of from the flange substantially coaxial with the axis. The remainder of the sleeve 16, corresponding generally to the distal end portion 20, has a generally conical shape when in the closed condition depicted in FIG. 1. As an example, the slits 26 extend from the end 30 of the distal end portion 20 through the sidewall 19 to the position 28 which can be located between the respective ends of the intermediate portion 34 of the sleeve 16. Those skilled in the art will appreciate that the intermediate portion 34 and distal end portion 20 may have other shapes than as shown and described herein.

The introducer apparatus 10 can be formed of a variety of materials including metals, alloys, polymers and/or composites, although it should be made of a material that is considered biocompatible or that can be made sufficiently biocompatible for at least temporary insertion into a desired tissue of a patient. Additionally, the flange 12 and sleeve 16 can be formed of the same or different materials. When formed of the same material, such as a plastic or thermoplastic material, the entire implanter apparatus can be injection molded from a common material. Different materials can also be utilized for the various parts of the apparatus 10.

The sidewall 19 of the sleeve 16 has a thickness that can be selected according to the material utilized for the sleeve to enable desired movement of the distal end portion to between the opened and closed conditions thereof. For instance, the sleeve 16, or at least the jaw members 24 thereof, can be formed of a flexible or pliant material to facilitate movement of the jaw members from the closed to open condition, such as upon insertion of an elongate member therethrough. The materials utilized might also be an inelastically deformable material so that jaw members 24 may remain in a substantially open condition after removal of the elongate device. No amount of resilience or memory of the jaw members or memory of the material is required for implementing the introducer apparatus 10.

Additionally, the introducer apparatus 10 can include means, such as an annular structure (e.g., a ring) 36, to inhibit movement of the distal end portion 20 from the closed condition to the open condition. The annular structure 36 also inhibits relative movement of the elongate member through the introducer apparatus 10. In FIGS. 1 and 2, the structure 36 is depicted as a ring mounted around an exterior of the distal end portion 20 of the sidewall 19 of the introducer apparatus 10. Those skilled in the art will understand and appreciate other shapes (C-shaped or U-shaped) and configurations of structures that can be applied to the sidewall 19 to implement the functions described herein as being performed by the structure 36.

In the example of FIG. 1, the annular structure 36 applies a radially inward force to help hold the jaw members 24 in the substantially closed position. As a result, an elongate member 40 generally is only insertable until the end 42 of the elongate member engages the interior surface of the distal end portion. By inhibiting insertion of the elongate member 40 through the apparatus 10, the annular structure 36 facilitates insertion of the combination of the elongate member and introducer apparatus into an anatomical structure of a patient. The ring structure 36 also operates to maintain the distal end portion 20 and jaw members 24 in a generally conical arrangement as it is urged into an anatomical structure for implantation.

As shown in FIG. 2, the structure 36 can rest in a groove (or in one or more slots or notches) 38 to maintain the structure 36 at a desired axial position along the exterior of the sidewall 19 of the introducer apparatus 10. The structure 36 can be fixed (e.g., by an adhesive or ultrasonic welding) to a portion of the sidewall 19 or it can hold its position due to frictional forces. The structure 36 can be formed of a plastic, metal, rubber (e.g., a rubber-band-like or O-ring structure) or other material that can be employed to apply radially inward force to the sidewall 19. Thus, the structure 36 can be applied to urge the jaw members 24 in a closed condition, as shown in FIG. 1.

FIG. 2 further depicts the elongate member 40 inserted through the passage of the apparatus 10 such that the respect jaw members 24 are in an open condition. As mentioned above, the jaw members 24 can be urged into the open condition by inserting the elongate member 40 through the opening associated with the inner periphery 14 of the flange 12 and axially through the passage defined by the sidewall 19 of the introducer apparatus 10. In the example of FIG. 2, the elongate member 40 has been inserted within the passage such that a distal end 42 of the elongate member protrudes beyond the end 30 of the distal end portion 20.

To facilitate insertion of the elongate member 40 through the distal end portion 20 of the introducer apparatus 10, as shown in FIG. 2, the radially inward force being applied by the structure 36 can be removed from the sidewall 19, such as by cutting or removing the part of the structure 36. The removal can be made through a slit or slot 52 formed in the flange 12 or by providing some mechanism for otherwise breaking the radially inward force applied thereby. When the structure 36 is cut, for example, the jaw members 24 can be more easily urged into their open condition so that the elongate member 40 can substantially freely move through the passage defined by the interior of the introducer apparatus 10.

The jaw members 24 thus can be urged into their open condition in response to an exterior surface 44 of the elongate member 40 engaging an interior portion of the sidewall 19, which engagement causes the jaw members 24 to deflect outwardly away from the central axis 22. The material employed for the jaw members 24 can exhibit resilience or some shape memory so that the jaw members return approximately to the closed condition after the elongate member 40 has been withdrawn from the passage defined by the sidewall 19. Alternatively, the jaw members, depending on the material, might be inelastically deformable to remain in a generally open condition upon removal of the elongate member 40.

In the example of FIG. 2, the elongate member 40 is in the shape of a cylindrical barrel. Those skilled in the art will understand and appreciate various shapes and configurations of elongate members can be utilized in combination with introducer apparatus 10. For example, the elongate member 40 can correspond to a portion of an implanter in which a prosthesis (e.g., heart valve, annuloplasty ring, stents, etc.) or other device, indicated schematically at 48, can be positioned for implantation. Alternatively, the elongate member 40 might be a trocar that is employed to provide a passage for accessing (e.g., laparoscopically, arthroscopically, endoscopically, or otherwise) anatomical features within a patient.

The exterior surface 44 of the elongate member 40 can also include indicia 46 that can be utilized to ascertain the length of the elongate member that has been inserted through the introducer apparatus 10. For instance, a proximal end of the introducer apparatus 10 (e.g., the inner periphery 14 of the flange 12) can align with a circumferentially extending indicia 46 to indicate a measurement as to how far the distal end 42 of the elongate member 40 has been inserted through the introducer apparatus, such as a distance beyond the flange 12. This can be used, for example, to position the end 42 of the elongate member 40 accurately relative to anatomical structures within the patient, such as in an organ or other implantation site.

Additionally or alternatively, the introducer apparatus 10 can include another structure 50 applied to the annular flange 12 to inhibit movement of the elongate member 40 axially through the passage defined by the sidewall 19. In the example of FIGS. 1 and 2, the structure 50 is depicted as a ring (e.g., an O-ring) applied to and engaging the outer periphery 18 of the flange 12. To facilitate holding the structure 50 to the flange 12, the outer periphery 18 can be recessed or include a groove around the flange.

One or more slits (or notches) 52 can also extend through the flange 12 to provide an adjustable cross-sectional dimension for the flange. The slits 52, for example, can extend at least from the outer periphery 18 to the inner periphery 14, and may further extend along a proximal part of the sidewall 19, such as shown in FIGS. 1 and 2. Thus, by applying the structure 50 to the outer periphery 18 of the flange 12, the slits 52 can be urged toward a closed condition (FIG. 1) so that the inner periphery 14 of the flange 18 exerts radially inward force along an exterior of the elongate member 40. This radially inward force (while the structure 50 is applied) inhibits axial movement of the introducer apparatus 10 relative to the elongate member 40. As a result, manipulation and insertion of the combination of the elongate member 40 and introducer apparatus 10 into anatomical structures can be facilitated, such as for implanting a prosthesis or other device. After the introducer apparatus 10 has been inserted so that the flange engages the structure, the structure 50 can be cut or removed from the flange (as shown in FIG. 2) to remove the radially inward force being applied. Thus, when the force being applied by the structure 50 has been removed, the elongate member 40 can move substantially freely through the apparatus 10 to facilitate positioning the distal end 42 at or near the desired implantation site.

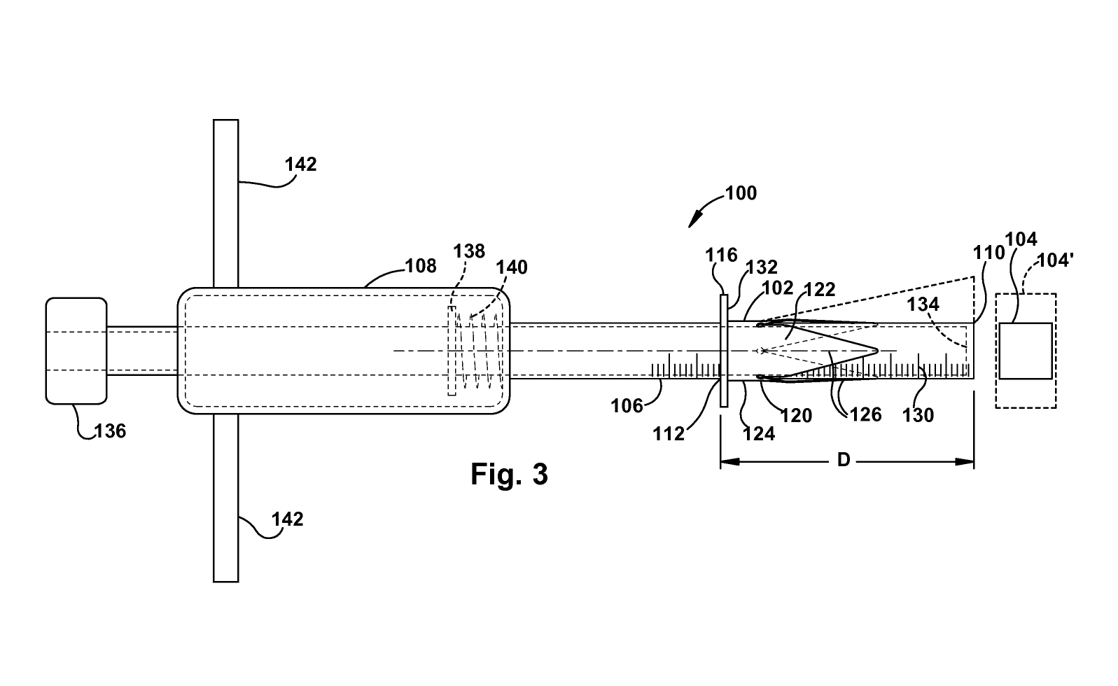

FIG. 3 illustrates an example of an implanter 100 in combination with an introducer apparatus 102 in accordance with an aspect of the present invention. The implanter/introducer combination 100, 102 can be employed for to facilitate low-invasive implantation of an article 104. Those skilled in the art will understand and appreciate various types of prostheses as well as other types of implantable devices that might be implanted using the implanter/introducer combination 100, 102 and, further that modifications to the implanter and/or introducer may be needed depending on the implantation site and the type of prosthesis or device 104.

In the example of FIG. 3, the implanter 100 includes an elongate cylindrical barrel 106 that extends from a body portion 108 and terminates in an open end 110. The barrel 106 has an inner diameter that may vary according to the type of article 104 as well as the dimension and configuration of the article being implanted. The introducer apparatus 102 can be slidably mounted for movement along an exterior of the barrel 106, such as by insertion of the end 110 of the barrel through an opening at a proximal end 112 of the introducer apparatus. The introducer 102 can be substantially similar to that shown and described with respect to FIGS. 1 and 2.

Briefly stated, the introducer apparatus 102 includes a flange portion 116 at the proximal end 112 of the introducer apparatus and a sidewall portion 120 extending longitudinally from the flange portion. The introducer apparatus 102 terminates in a distal portion 122 spaced apart from the flange 116 by an intermediate portion 124. The distal portion 122 includes one or more members, such as jaw members 126, which are moveable relative to each other. The jaw members 126 can be moveable at least from a closed condition to an open condition and, depending on the material, also from the open condition back to the closed condition.

In the example of FIG. 3, the introducer apparatus 102 is slidably positioned for movement along the barrel 106 of the implanter 100. The introducer apparatus 102 may be removable from the barrel 106. Alternatively, the end 110 of the barrel 106 can include a retaining feature (e.g., a rib, a tab, thicker sidewall portion, etc. (not shown)) to help prevent removing the introducer apparatus 102 completely from the barrel.

Similar to the example of FIG. 2, the introducer apparatus 102 is depicted in FIG. 3 in an opened condition, in which an exterior surface of the barrel 106 engages in inner surface of the sidewall 120 of the introducer apparatus. In particular, the inner surface of the sidewall 120 engages the exterior surface of the barrel 106. The engagement between the barrel 106 and the introducer apparatus 102 urges the respective jaws 126 of the introducer apparatus to the open condition. In the open condition, the distal end portion 122 of the introducer apparatus 102 has a generally crown-like appearance, in which adjacent side edges of the respective jaw members 126 are spaced apart from each other in generally V-shaped voids.

As described with respect to FIG. 3, the barrel 106 of the implanter 100 can include indicia 130 disposed along the exterior surface of the barrel between the distal end 110 of the barrel. The indicia 130 can be printed or otherwise marked onto the barrel 106 as a scale or ruler. In this way, the proximal end 112 of flange portion 116 can align with adjacent indicia 130 to identify a measure of the distance indicated at D between the flange portion 116 and the distal end 110 of the barrel 106. The scale or ruler markings that form the indicia 130 further can be adjusted to accommodate for the thickness of the flange portion 116, such that the measurement can correspond to the distance from a distal side 132 of the flange 116 to the end 110 of the barrel 106.

In FIG. 3, the prosthesis or device 104 is illustrated as being discharged from the implanter 100. For example, the implanter 100 includes a plunger or other means 134 for discharging the prosthesis or device 104 from the end 110 of the barrel 106. The implanter 100 can include a flange or other feature 138 operatively connected with the plunger 134. A biasing element (e.g., one or more springs) 140 is operative to urge the plunger 134 axially away from the discharge end 110. Thus, the plunger 134 can be urged or activated for axial movement through the barrel by employing a knob 136 that is operatively connected (directly or indirectly) with the plunger accordingly. For instance, a user can push the knob 136 with the user's thumb while holding a handle or flange 142 with the user's index finger and middle finger (e.g., similar to using a syringe). Other means (e.g., trigger, spring activated, threads, etc.) can be employed for moving the plunger 134 in a desired direction. Additionally, after discharging the prosthesis 104 from the barrel 106, the plunger 134 may be removed so that the barrel provides a passage through which a corresponding implantation site (near the end 110) and the implanted prosthesis can be accessed.

Those skilled in the art will understand and appreciate various types of prostheses and implantable devices 104 that can be implanted via an implanter/introducer combination 100, 102. The implantable article 104 can have a fixed cross-sectional dimension, as shown by a solid line at 104. Alternatively, the device can be expandable to an enlarged cross-sectional dimension, such as indicated by dashed line at 104′.

By way of further example, to facilitate insertion of an expandable type of the article 104′ into the barrel 106, the prosthesis or device should be deformed to a reduced cross-sectional dimension, such as about less than an internal dimension of the barrel. As an example, the inner diameter of the barrel 106 can range from about 5 mm to about 15 mm, whereas the outer diameter of the article 104′ (in its expanded condition) might range from about 15 mm to about 35 mm. Thus, the barrel 106 can accommodate a prosthesis 104′, which has been deformed to reduce cross-sectional dimension, without compromising the durability and operation of the prosthesis. Alternatively, as mentioned above, the inner diameter of the barrel 106 can approximate the outer diameter of the prosthesis 104′, such as when the prosthesis does not easily compress.

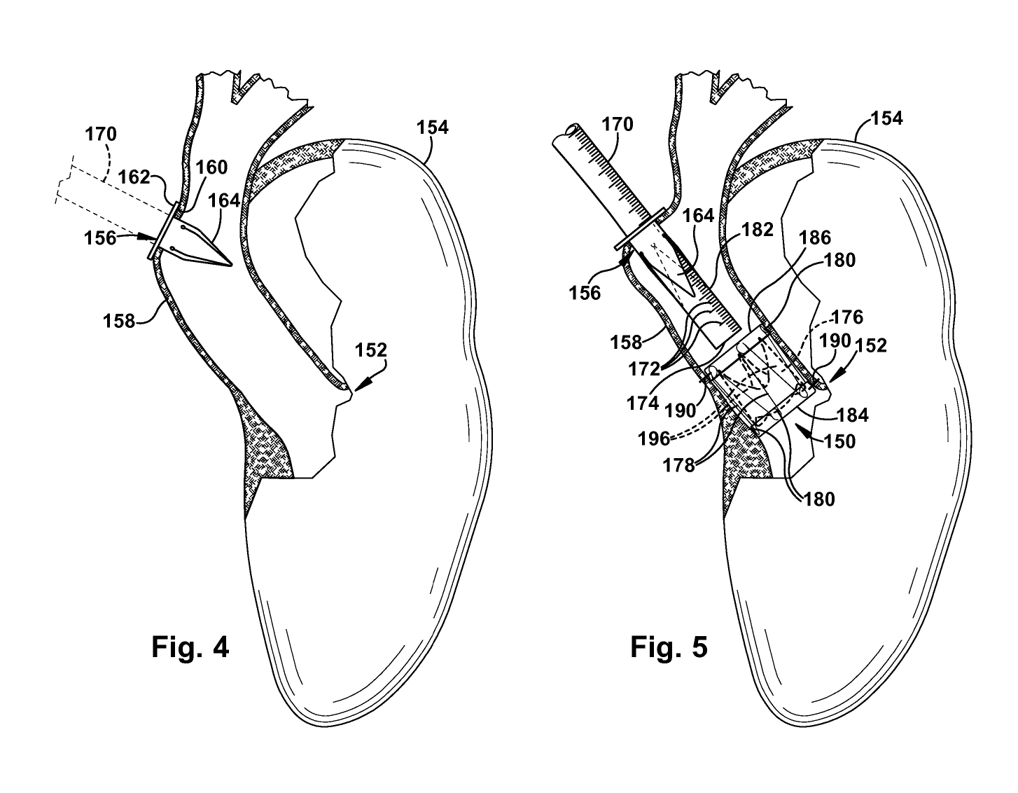

FIGS. 4 and 5 depict an example of a portion of a procedure that can be utilized for implanting a heart valve prosthesis 150 at an aortic annulus 152 of a patient's heart 154. In the example of FIGS. 4 and 5, it is assumed that at least portions of a defective valve or the entirely defective valve that is being replaced has been removed from the aortic position. For example, optional sutures (not shown) may be applied (through the barrel) at a location near the inflow end 184 and/or at the outflow end 186. Those skilled in the art will understand and appreciate that some heart valves might alternatively be implanted while the defective valve remains intact. Additionally, as described herein, different types of implantable articles might be implanted in a similar way to improve operation of a defective valve.

In FIG. 4, an introducer apparatus 156 has been inserted into the aorta 158. As an example, an incision is made at a desired location in the aorta 158 of the patient's heart 154 and the introducer apparatus 156 is inserted into the incision, such that a distal side 160 of a flange 162 engages the exterior aortic wall 158. A purse string (not shown) can be applied around the insertion to mitigate bleeding by tightening the heart muscle tissue around the sidewall of the introducer apparatus 156. The introducer apparatus 156 can be inserted separately or in combination with the implanter 170 while the implanter is inserted partially into the introducer apparatus. Once the introducer apparatus 156 has been inserted, as shown in FIG. 4, a distal end portion 164 of the introducer apparatus extends into the aorta 158 in the closed condition. The introducer apparatus 156 can be constructed according to any of the types described herein, such as shown and described in FIGS. 1-2 or include any combination of the features of FIGS. 1-2. Those skilled in the art will understand and appreciate that differently sized and configurations of introducer apparatuses can be utilized, for example, depending upon the location in which such apparatuses are to be implanted and the dimensions and configuration of the implanter 170. Additionally, the introducer apparatus 156 can be inserted into other anatomical structures, including other blood vessels (e.g., pulmonary artery), to provide access to an intended implantation site.

In the example of FIG. 4, the introducer apparatus 156 is inserted into a position such that a generally direct path can be provided from the introducer apparatus to the desired implantation site (e.g., the aortic annulus 152) for implanting the prosthesis 150. An O-ring or other means (not shown) can be applied to a sidewall of the introducer apparatus 156 to help hold the jaws closed during its insertion into the heart 154 as well as to inhibit passage of the barrel of the implanter 170 through the distal end of the introducer apparatus. The path from the introducer apparatus 156 to the implantation site 152 can be substantially linear or it can provide a substantially curved or indirect path depending upon the type of implanter being utilized.

FIG. 5 depicts an example of the heart valve prosthesis 150 being implanted at the aortic annulus 152 by employing an implanter 170 in combination with the introducer apparatus 156. Those skilled in the art will understand and appreciate various types of implanters 170 that can be utilized in conjunction with an introducer apparatus based on the teachings contained herein. Furthermore, it will be appreciated that the dimensions and configurations of the introducer apparatus 156 can be adapted according to the dimensions and configuration of the implanter 170, or the dimensions and configurations of the implanter can be adapted according to the dimensions and configurations of the introducer apparatus.

As described herein, indicia 172 along the sidewall of the implanter 170 provide a means for determining a measure of the distance that an end 174 of the implanter has been inserted to within the aorta 158. Thus, the measurement information from the indicia 172 can facilitate discharging the prosthesis 150 from the implanter 170 at a desired implantation site 152. The distance and location of the implantation site 152 can be ascertained by employing a number of techniques. For example, the end 174 of the implanter 170 can be positioned via ultrasonic or radiographic means, such as a cardiac 3-D echo performed before and/or during the implantation procedure.

It is to be appreciated that the prosthesis 150 may be implanted at the aortic annulus 152 during a conventional open chest procedure or during a closed chest procedure. Because the only incision is in the patient's aorta 158, the implantation can be performed during very short open chest surgery, for example, with reduced cardiopulmonary bypass when compared to conventional procedures.

However, it is to be understood and appreciated that if the patient has a calcified aortic valve, the patient typically will be put on cardiopulmonary bypass to remove the defective valve or at least calcified portions thereof and to implant the prosthesis 150. Advantageously, a prosthesis 150 may still be implanted in accordance with the present invention more effectively than many conventional approaches even when cardiopulmonary bypass is utilized.

In the example of FIG. 5, the heart valve prosthesis 150 is depicted as a valve that can be implanted without requiring sutures (e.g., referred to as a sutureless valve), although sutures can be used. The prosthesis 150 includes a heart valve 176 mounted within a support 178 that is formed of a material that helps maintain a generally cylindrical configuration for the implanted valve 176.

According to one aspect of the present invention, the support 178 can be formed from a shape memory material, such as Nitinol. For example, the support 178 can be formed from a small cylindrical tube of the shape memory material, such as via a laser cutting (ablation) process in which the desired sinusoidal sidewall is cut from the tube. Associated spikes 180 can be formed as an integrated structure having a desired shape and size to extend generally outwardly and arcuately form the respective ends of the support 178. Additionally, ends of the spikes 180 can have tapered or sharpened tips (with or without barbs) to facilitate gripping surrounding tissue of the aorta 158 when implanted in a sutureless implantation. Additionally or alternatively, some sutures can be utilized in combination with the spikes 180 to retain the prosthesis 150 at a desired position. For instance, sutures 190 can be applied externally to prosthesis 150 after its implantation or sutures can be applied internally, such as through a passage defined by the implanter 170.

When the support 178 is formed of a shape memory material, such as Nitinol, the support and prosthesis 150 can be reduced to a reduced cross-section for insertion into a barrel 182 of the implanter 170. For instance, the support 178 can be heated to its transformation temperature and forced to a desired cross-sectional dimension and configuration (austenitic) form, corresponding to an expanded configuration of the support. The support 178 can then be bent or deformed to a reduced cross-sectional dimension when in its low-temperature (martensitic) form to facilitate its mounting the prosthesis 150 within a barrel 182 of the implanter 170, for example. When the prosthesis 150 is implanted and discharged from the barrel 182, the support 178 returns to its austenitic form, such as shown in FIG. 5.

The valve 176 can be a biological valve or a mechanical valve. For the example of a biological type of valve 176, the valve can be a homograft or xenograft, or it can be manufactured from a biological tissue material to include one or more leaflets arranged for providing substantially unidirectional flow of blood through the valve. The valve 176 includes an inflow end 184 and an outflow end 186 at axially opposed ends of the valve, with a sidewall portion extending between the ends thereof. The inflow end 184 of the valve 176 is positioned near a corresponding inflow end of the support 178. The prosthesis 150 can also include sidewall portion, which can be a tubular valve wall, such as for a homograft or xenograft valve 176. A plurality of leaflets 196 extend radially inward from the valve wall and coapt along their adjacent edges to provide for substantially unidirectional flow of blood through the valve 176. The valve 176 can be connected within the support 178 via sutures or other known connecting means, for example.

For example, when being implanted at an aortic position, an aortic valve (e.g., equine, porcine, bovine, etc.) can be utilized for the valve portion 176 of the prosthesis 150, although other types of valves could also be used. It is to be understood and appreciated that various types of valves 176 could be employed to provide the heart valve prosthesis 150 in accordance with an aspect of the present invention.

As another example, the valve 176 can include one or more leaflets mounted within a length of tubular valve wall or other generally cylindrical biocompatible material and operate in a known manner to provide for the unidirectional flow of fluid through the valve from the inflow to outflow ends 184, 186. Examples of prostheses and valves include those shown and described in U.S. Pat. Nos. 5,935,163, 5861,028 or 5,855,602, as well as others mentioned herein or otherwise known in the art. Those skilled in the art will further understand and appreciate that unstented, as well as stented, valves can be implanted by employing an introducer apparatus/implanter combination.

By way of further example, the valve 176 and any other biological material employed to provide the prosthesis 150 can be formed from natural biological material, such as a natural tissue sheets (e.g., animal pericardium, dura matter and the like), although other natural or synthetic biocompatible materials (e.g., molded collagen) also could be utilized. For instance, the biological materials can be cross-linked with glutaraldehyde and undergo a detoxification process with heparin bonding, such as according to a NO-REACT® treatment process available from Shelhigh, Inc. of Union, N.J. The NO-REACT® treatment improves biocompatibility of the valve 176 and mitigates calcification and thrombus formation.

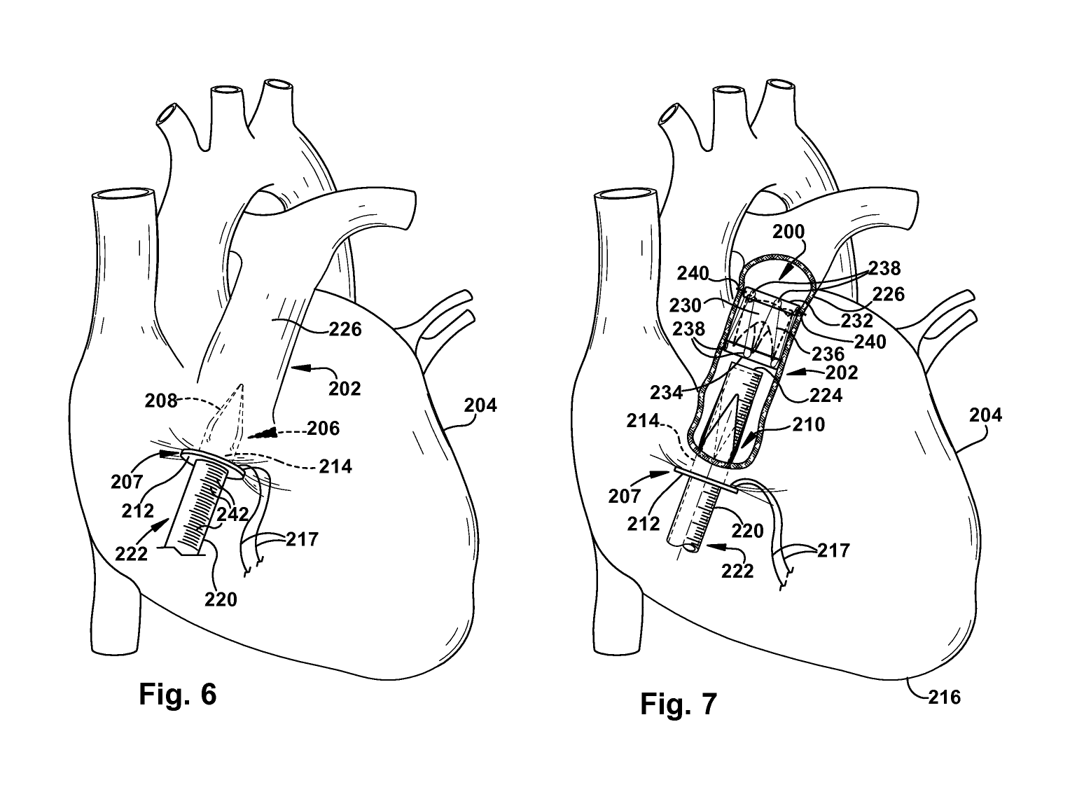

FIGS. 6 and 7 depict another example approach that can be utilized for implanting a heart valve prosthesis 200 according to an aspect of the present invention. In the example of FIGS. 6 and 7, the prosthesis 200 is implanted at a pulmonic position 202 of a patient's heart 204. Identical reference numbers are used in FIGS. 6 and 7 to identify corresponding features.

In the example of FIG. 6, an introducer apparatus 206 has been inserted into an anterior wall 207 of the right ventricle 210 (FIG. 7) of the heart 204, such that a distal end portion 208 of the introducer apparatus extends into the right ventricle of the heart. The insertion location into the heart 204 can vary to provide a substantially direct path to the implantation site at the pulmonic position 202. A flange portion 212 of the introducer apparatus 206 engages the exterior surface of the anterior wall 207 of the right ventricle 210. An intermediate portion 214 of the introducer apparatus 206 thus is surrounded by the heart muscle 204 at the anterior wall 207 of the heart. The engagement of the surrounding heart muscle 204 with the introducer apparatus 206 can be facilitated, for example, by applying a double purse string suture 217 generally around the insertion location, such as prior to making an incision through the heart 204 into the right ventricle 210.

The distal end portion 208 is in the closed condition during insertion of the introducer apparatus 206 into the heart muscle 204. The closed condition of the distal end portion 208 can be facilitated by employing a ring or other structure, such as shown and described in FIGS. 1-2. The location in which the introducer apparatus 206 is inserted into the heart 204 generally will depend on the type of implanter and the location of the implantation site.

In the example of FIGS. 6 and 7, a barrel 220 of an implanter 222 is depicted as being inserted into the introducer apparatus 206 and into the heart 204. The barrel 220 is substantially linear barrel, such as part of a catheter system or other type of implanter described herein. Those skilled in the art will understand and appreciate that a flexible and/or curved barrel 220 can be utilized. The barrel 220 is inserted at a position to provide a substantially linear or direct path for implanting the heart valve prosthesis 200 at the desired pulmonic position 202. In FIG. 7, a distal end 224 of the barrel 220 has been inserted through the introducer apparatus 206 that so that respective members of the distal end portion 208 are urged apart to the open condition by the barrel 220 and thereby permit substantially free movement of the barrel through the introducer apparatus.

By inserting the introducer apparatus 206 through the anterior wall 207 proximal the pulmonary artery 226, a substantially direct or generally linear implantation of the prosthesis 200 can be performed with little or no cardiopulmonary bypass. As mentioned above, however, when the patient's defective valve is calcified, cardiopulmonary bypass will typically be performed for removing the calcified valve portions prior to implantation. The removing of the valve can be performed, for example, by employing a trocar or other tube inserted through the introducer apparatus 206 or during an open heart procedure prior to implanting the prosthesis 200.

Those skilled in the art will understand and appreciate other possible paths through the heart or associated arteries or veins that could be employed for positioning the distal end 224 of the barrel 220 to enable implantation of the prosthesis 200 in accordance with an aspect of the present invention.

The barrel 220 further can include indicia 242, such as corresponding to ruler markings, for providing an indication of measurement as to the distance which the end 224 of the barrel has been inserted into the heart 204. Accordingly, the measurement from the indicia 242 enables a user to discharge the prosthesis 200 into the pulmonary artery 226 at the desired implantation site, namely at the pulmonic position 202. Once at the desired position, the prosthesis 200 can discharged or ejected from the barrel 220 into the outflow of the right ventricle 210, as illustrated in FIG. 7.

The prosthesis 200 can be substantially similar to that shown and described with respect to FIG. 5, although other types of valves can also be utilized. Briefly stated, the prosthesis 200 includes a valve 230 having an inflow end 232 and an outflow end 234 spaced therefrom. The valve 230 can be a natural tissue heart valve, such as a homograft or xenograft, although other types of biological tissue manufactured heart valves also could be utilized. Still further, the valve 230 can be a mechanical or biomechanical type of valve. The valve 230 can be mounted within a support or stent 236, such as one of the types described herein, or the valve could be unstented. Since the prosthesis 200 is implanted at the pulmonic position 202, which is exposed to lower blood pressure, a more pliant valve 230 generally can be utilized as compared to when implanting a prosthesis at the aortic position (FIG. 5).

For a sutureless type of implantation, the support 236 can include spikes or protruding portions 238 for engaging surrounding tissue of the pulmonary artery 226 in its implanted position. The spikes or protruding portions 238 thus inhibit axial and/or angular movement of the implanted prosthesis 200. While the spikes or protruding portions 238 are depicted as extending generally arcuately from the respective end portions, it will be appreciated that suitable spikes or protruding portions can be located at any external location of the support 236. It will be appreciated, however, that sutures can also or alternatively be employed as a means to secure the valve relative to the pulmonary artery 226. For instance, sutures 240 can be applied externally to prosthesis 200 after its implantation, or sutures can be applied internally, such as through a passage defined by the barrel 220.

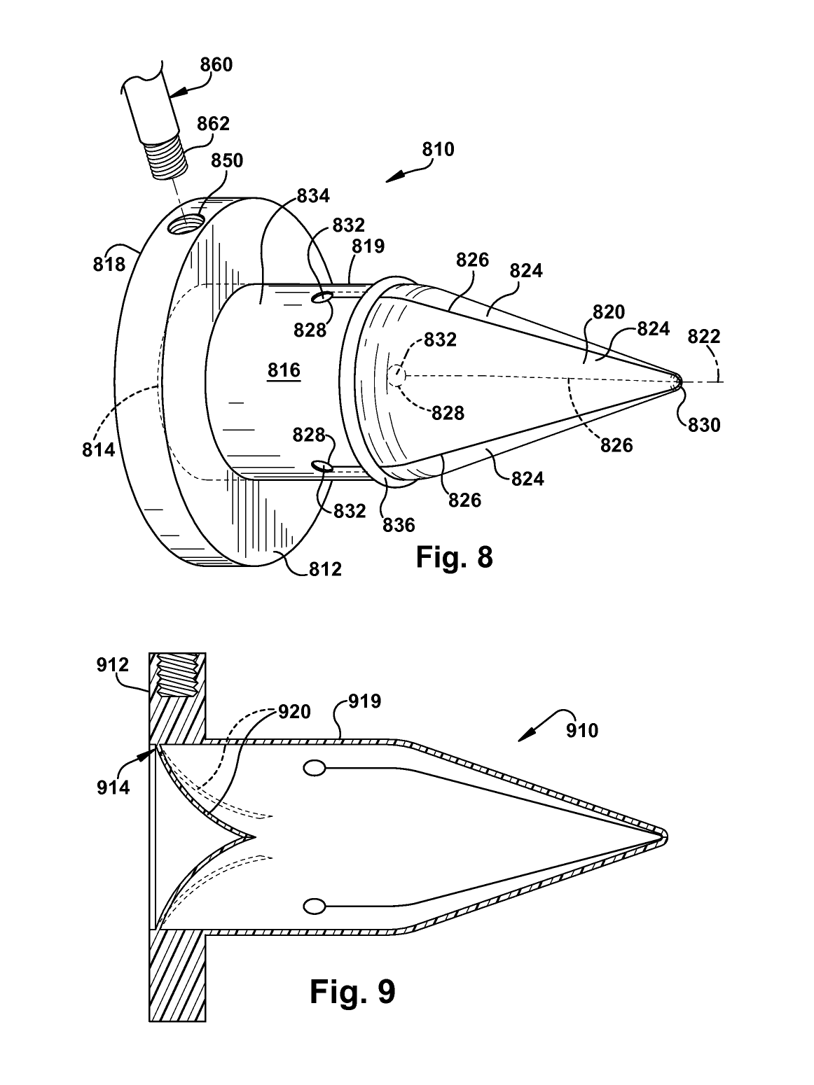

FIG. 8 depicts another example of an introducer apparatus 810 that can be implemented in accordance with an aspect of the present invention. The introducer apparatus 810 includes a flange 812. While the flange 812 is illustrated as a complete annular flange, those skilled in the art will understand and appreciate that the flange 812 can be implemented in a variety of shapes (e.g., rectangular hexagonal, etc.) and that flange 812 need not extend in a complete annular structure. For example, the flange 812 can be implemented as substantially co-planner tabs, a rim, as well as a curved or c-shaped flange portion or as a thicker sidewall portion of the apparatus 810.

In the example of FIG. 8, the flange 812 includes an inner periphery 814 that defines an opening into an associated sleeve 816. While the inner periphery is 814 generally circular in the example of FIG. 8, it can have other shapes. The flange 812 also includes an outer periphery 818 spaced apart from the inner periphery 814 by an associated intermediate portion thereof.

The sleeve 816 has a sidewall 819 that extends longitudinally from the flange 812 and terminates in a distal end portion 820. A central axis 822 extends through a center of the sleeve 816 and through the opening defined by the inner periphery 814 of the flange 812. An adjustable opening is operatively associated with the distal end portion 820. The adjustable opening includes means movable from at least a closed condition (FIG. 8) to an open condition (similar to FIG. 2) to permit substantially free movement of an article, such as an implanter or trocar, axially through a passage defined by the sidewall 819.

By way of example, the adjustable opening in the distal end portion 820 can include two or more jaw members 824 that are movable in a generally radially direction relative to the axis 822 between the open and closed conditions. In the example of FIG. 8, the distal end portion 820 includes a plurality of three jaw members 824 positioned in closed condition. Longitudinally extending side edges of adjacent jaw members 824 define longitudinally extending slits 826. The longitudinally extending slits 826 extend through the sidewall 819 from a first position 828, which is located proximal to the flange 812, to intersect an opposed end 830 of the distal end portion 820 to separate the jaw members 824. An aperture 832 further may extend through the sidewall 819 of the sleeve 816 at the first position 828 of each of the respective slits 826. The respective apertures 832 operate as hinges to facilitate the generally radial movement of the respective jaw members 824 from the closed condition to the open condition. Other structure, such as hinges, can also be employed to provide for movability of the jaw members 824. Additionally, one of the slits 826 may extend completely through the sidewall 819 as well as through the flange 812 to accommodate differently sized elongate members within the passage. Thus, the sidewall 819 can be generally cylindrical (such as shown in FIG. 8, or the sidewall can be generally C-shaped.

In the example of FIG. 8, the sleeve 816 can include an intermediate portion 834 located between the flange 812 and the distal end portion 820. The intermediate portion 834 of the sleeve 816 can have a generally right circular cylindrical cross section that extends a predetermined length of from the flange 812 substantially coaxial with the axis 822. The remainder of the sleeve 816, corresponding generally to the distal end portion 820, has a generally conical shape when in the closed condition depicted in FIG. 8. As an example, the slits 826 extend from the end 830 of the distal end portion 820 through the sidewall 819 to the position 828 which can be located between the respective ends of the intermediate portion 834 of the sleeve 816. Those skilled in the art will appreciate that the intermediate portion 834 and distal end portion 820 may have other shapes than as shown and described herein.

The introducer apparatus 810 can be formed of a variety of materials including metals, alloys, polymers and/or composites, although it should be made of a material that is considered biocompatible or that can be made sufficiently biocompatible for at least temporary insertion into a desired tissue of a patient. Additionally, the flange 812 and sleeve 816 can be formed of the same or different materials. When formed of the same material, such as a plastic or thermoplastic material, the entire implanter apparatus can be injection molded from a common material. Different materials can also be utilized for the various parts of the apparatus 810.

The sidewall 819 of the sleeve 816 has a thickness that can be selected according to the material utilized for the sleeve to enable desired movement of the distal end portion 820 to between the opened and closed conditions thereof. For instance, the sleeve 816, or at least the jaw members 824 thereof, can be formed of a flexible or pliant material to facilitate movement of the jaw members from the closed to open condition, such as upon insertion of an elongate member therethrough. The materials utilized might also be an inelastically deformable material so that jaw members 824 may remain in a substantially open condition after removal of the elongate member. No amount of resilience or memory of the jaw members 824 or memory of the material is required for implementing the introducer apparatus 810.

Additionally, the introducer apparatus 810 can include means, such as an annular structure (e.g., a ring) 836, to inhibit movement of the distal end portion 820 from the closed condition to the open condition. The annular structure 836 also inhibits relative movement of the elongate member through the introducer apparatus 810. In FIG. 8, the structure 836 is depicted as a ring mounted around an exterior of the distal end portion 820 of the sidewall 819 of the introducer apparatus 810. Those skilled in the art will understand and appreciate other shapes (C-shaped or U-shaped) and configurations of structures that can be applied to the sidewall 819 to implement the functions described herein as being performed by the structure 836.

In the example of FIG. 8, the annular structure 836 applies a radially inward force to help hold the jaw members 824 in the substantially closed position. As a result, an elongate member generally is only insertable until an end of the elongate member engages the interior surface of the distal end portion 820. By inhibiting insertion of an elongate member through the introducer apparatus 810, the annular structure 836 facilitates insertion of the combination of the elongate member and introducer apparatus into an anatomical structure of a patient. The ring structure 836 also operates to maintain the distal end portion 820 and jaw members 824 in a generally conical arrangement as it is urged into an anatomical structure for implantation.

The jaw members 824 thus can be urged into their open condition in response to an exterior surface of the elongate member engaging an interior portion of the sidewall 819, which engagement causes the jaw members 824 to deflect outwardly away from the central axis 822. The material employed for the jaw members 824 can exhibit resilience or some shape memory so that the jaw members return approximately to the closed condition after the elongate member has been withdrawn from the passage defined by the sidewall 819. Alternatively, the jaw members 824, depending on the material, might be inelastically deformable to remain in a generally open condition upon removal of the elongate member, as described with respect to FIGS. 1 and 2.

In the example of FIG. 8, an axial thickness of the flange 818 may be 2-3 times greater than the axial thickness of the flange 18 of the introducer apparatus 10 of FIG. 1. This allows the flange 818 to include an internally threaded aperture 850. As shown in FIGS. 8, 13 and 14, a threaded end 862 of a handle member 860 may be secured to the flange 818 in order to facilitate guiding the introducer apparatus 810 during insertion into the heart muscle 204. The internally threaded aperture 850 may extend up to the interior of the sidewall 819 in order to better secure the handle member 860. The materials used for construction of the flange 818 and the handle member 860 may alternatively be higher strength so that the dimensions of the flange 818 and the handle member 860 may be similar to the flange 18 of the introducer apparatus 10 of FIG. 1 while still having the handle feature of FIGS. 8-16.

FIG. 9 depicts a cross-sectional view of an example of an introducer apparatus 910 that can be implemented in accordance with another aspect of the present invention. The introducer apparatus 910 is similar to the introducer apparatus 810 of FIG. 8 and also includes a check valve structure 920 for preventing backflow of fluid when a plug or other member, such as the elongate member 40 in FIG. 2, is not present in the passage defined by a side wall 919 of the introducer apparatus 910, as in FIG. 9. The valve member 920 may include a single flap member (not shown) shaped for blocking the passage defined by the side wall 919 and preventing backflow of fluid. Alternatively, the valve member 920 may comprise two or more flap members (FIG. 9) defining overlapping arcs for preventing backflow of fluid. The flap members resiliently engage each other such that the flap members re-engage each other when the elongate member is withdrawn from the passage.

The valve member 920 may be molded to an inner periphery 914 of a flange 912 of the introducer apparatus 910 adjacent the flange. Alternatively, the valve member may be axially offset from the flange (not shown). The valve member 920 may be constructed of rubber or other suitable material resilient enough to spring back into the closed position of FIG. 9. The valve member 920 may be formed of the same or a different material from the material that forms the sidewall 919 of the apparatus 910.

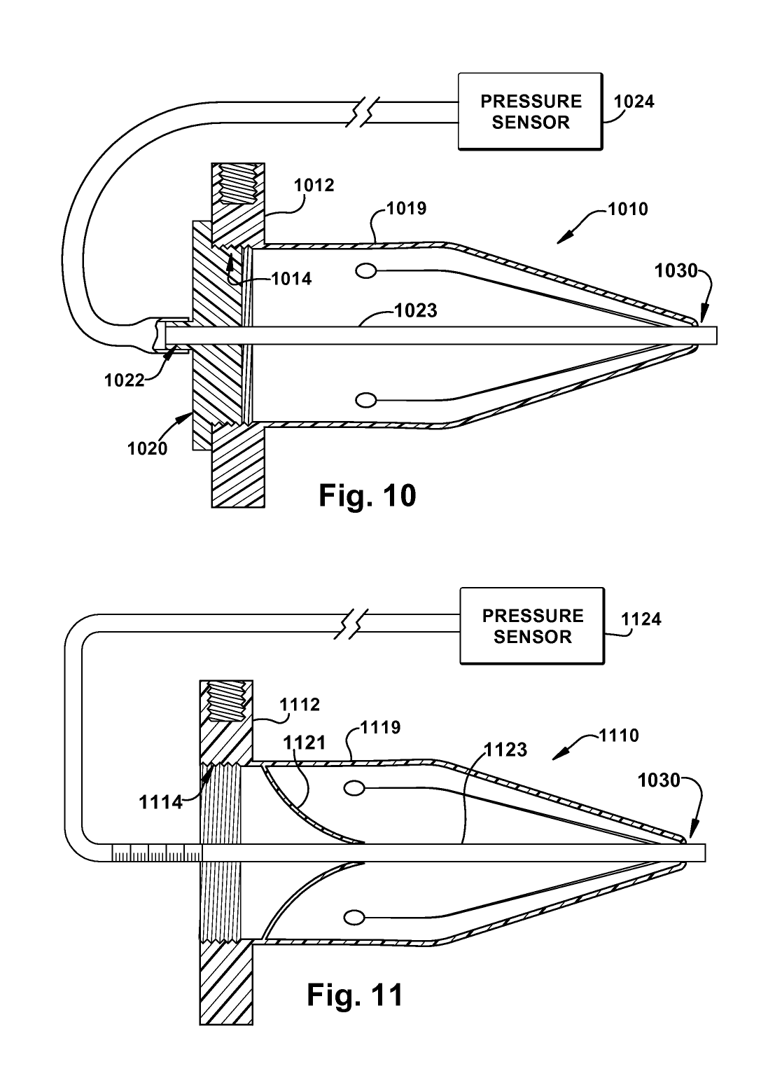

FIG. 10 depicts another example of an introducer apparatus 1010 that can be implemented in accordance with another aspect of the present invention. The introducer apparatus 1010 is similar to the introducer apparatus 810 of FIG. 8 and also includes an externally threaded and solid (not shown) flow obstructing structure 1020 for preventing flow of fluid in either axial direction when an elongate member (i.e., elongate member 40 in FIG. 2) is not present in the passage defined by a side wall 1019 of the introducer apparatus 1010, as in FIG. 10. The structure 1020 may be a plug threaded into mating threads of an inner periphery 1014 of a flange 1012 of the introducer apparatus 1010. The structure 1020 may be constructed of a similar material as that of the introducer apparatus 1010 or other suitable material.

Alternatively, an unthreaded valve member 1020 (not shown), along with an unthreaded inner periphery of the flange, may together form an interference fit thus securing the valve member 1020 to the flange 1012. A resilient and unthreaded valve member 1020 may also be tapered (not shown) to act as a stopper or a cork to form a friction fitting and thus prevent flow of fluid in either axial direction when an elongate member is not present in the passage defined by a side wall 1019 of the introducer apparatus 1010

The valve member 1020 may also include a centrally disposed sensor port 1022 and an elongate tube 1023 for facilitating a connection to a pressure sensor 1024 for monitoring the pressure within the passage defined by the side wall 1019 of the introducer apparatus 1010 (FIG. 10). The elongate tube 1023 may extend beyond a distal end 1030 of the introducer apparatus 1010. For example, a distal end 1026 of the tube can extend through the apparatus 1010 beyond the distal end portion of the sleeve. The pressure sensor 1024 can in turn monitor the pressure within the patient' heart, which may vary according to the position of the distal end 1026 of the tube 1023.

FIG. 11 depicts another example of an introducer apparatus 1110 that can be implemented in accordance with another aspect of the present invention. The introducer apparatus 1110 is similar to the introducer apparatus 1010 of FIG. 10. The introducer apparatus 1110 may further include a check valve structure 1121, similar to the structure 920 of FIG. 9, for preventing backflow of fluid when a plug or other member, such as the elongate member 40 in FIG. 2, is not present in the passage defined by a side wall 1119 of the introducer apparatus 1110. The structure 1121 is axially offset from the flange 1112 so that the structure does not interfere with the threads of the inner periphery 1114.

The introducer apparatus 1110 further includes an elongate tube that can be connected to a pressure sensor 1124 for monitoring the pressure. The elongate tube 1123 can be inserted within and extend through the passage defined by the side wall 1119 of the introducer apparatus 1110 (FIG. 11). The inner periphery 1114 of the flange 1112 can include threads for receiving a plug (not shown), similar to the introducer apparatus 1010 of FIG. 10. The elongate tube 1123 may extend beyond a distal end 1130 of the introducer apparatus 1110 and through the valve structure 1121, such as shown in FIG. 11. Thus, the valve structure 1121 can prevent back flow of fluid through the apparatus when the elongate tube 1123 is present (FIG. 11) in the passage defined by a sidewall 1119 of the introducer apparatus 1110. The valve structure may also operate to prevent back flow of fluid through the sidewall of the apparatus in the absence of the elongate tube 1123.

As an example, the valve structure 1121 may include a single flap member (not shown) shaped for blocking the passage defined by the side wall 1119 and preventing backflow of fluid. Alternatively, the structure 1121 may comprise two or more flap members defining overlapping arcs for preventing backflow of fluid. The structure 1121 may be molded to an inner periphery 1114 of a flange 1112 of the introducer apparatus 1110 adjacent the flange (not shown) or axially offset from the flange (FIG. 11). The structure 1121 may be constructed of rubber or other suitable material resilient enough to spring back into a closed position, similar to FIG. 9.

A portion of the elongate tube 1123 can also include a printed scale (e.g., in metric or English units of measurement) or indicia, indicated at 1128. The scale or indicia 1128 can be employed to determine a relative position of the distal end 1126 of the tube. For example, the scale can establish a zero distance measurement to correspond to the distal end of the elongate tube 1126. The scale thus can be utilized to determine how far the tube has been inserted through the introducer apparatus 1110. Since the distal end of the tube can also be employed to monitor pressure (via the pressure sensor 1124), the measured distance of the distal end can be recorded when a change in pressure occurs so as to identify and/or confirm the position of patient's heart valve. That is, the detected change in pressure can be employed to determine and/or confirm how far and the direction that a corresponding implanter needs to be inserted through the introducer apparatus 1110 for implanting a cardiac prosthesis, such as described herein.

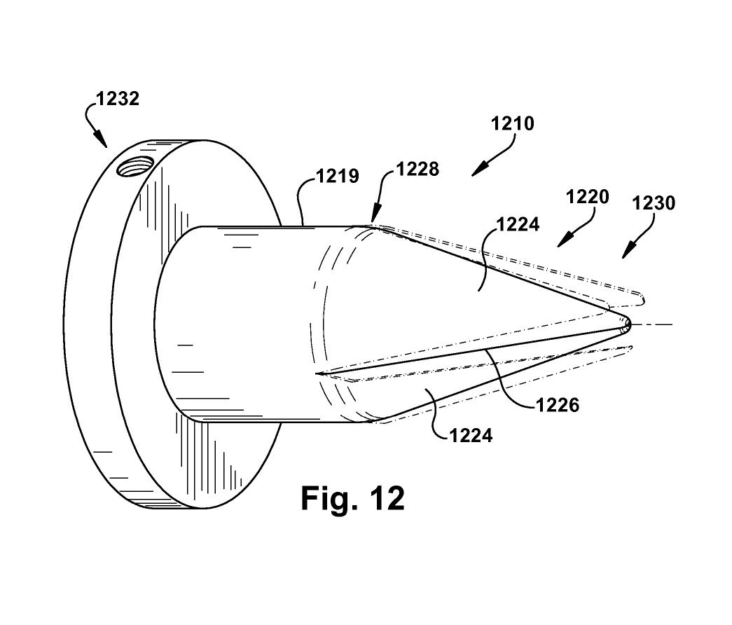

FIG. 12 depicts another example of an introducer apparatus 1210 that can be implemented in accordance with another aspect of the present invention. The introducer apparatus 1210 is similar to the introducer apparatus 810 of FIG. 8 and also includes a valve structure 1220 for preventing backflow of fluid when an elongate member (i.e., elongate member 40 in FIG. 2) is not present in the passage defined by a side wall 1219 of the introducer apparatus 1210, as in FIG. 12. The valve member 1220 is defined by jaw members 1224 constructed of rubber or other suitable resilient material such that the jaw members spring back into the closed position of FIG. 12 when an elongate member or pressure sensor tube is withdrawn from the passage defined by a side wall 1219 of the introducer apparatus 1210. In order to achieve this, slits 1226 only extend from an end 1230 of the introducer apparatus 1210 to an end 1228 of the conical portion defined by the jaw members 1224 (instead of extending further to the cylindrical sidewall 1219). As a result, the jaw members 1224 may engage each other along edges defined by the slits 1226 to seal the exterior of a distal end portion 1230 of the introducer apparatus 1210 from the interior of a proximal end portion 1232 of the introducer apparatus. That is, the jaw members themselves can be implemented to define a valve to prevent back flow of fluid through the introducer apparatus 1210.

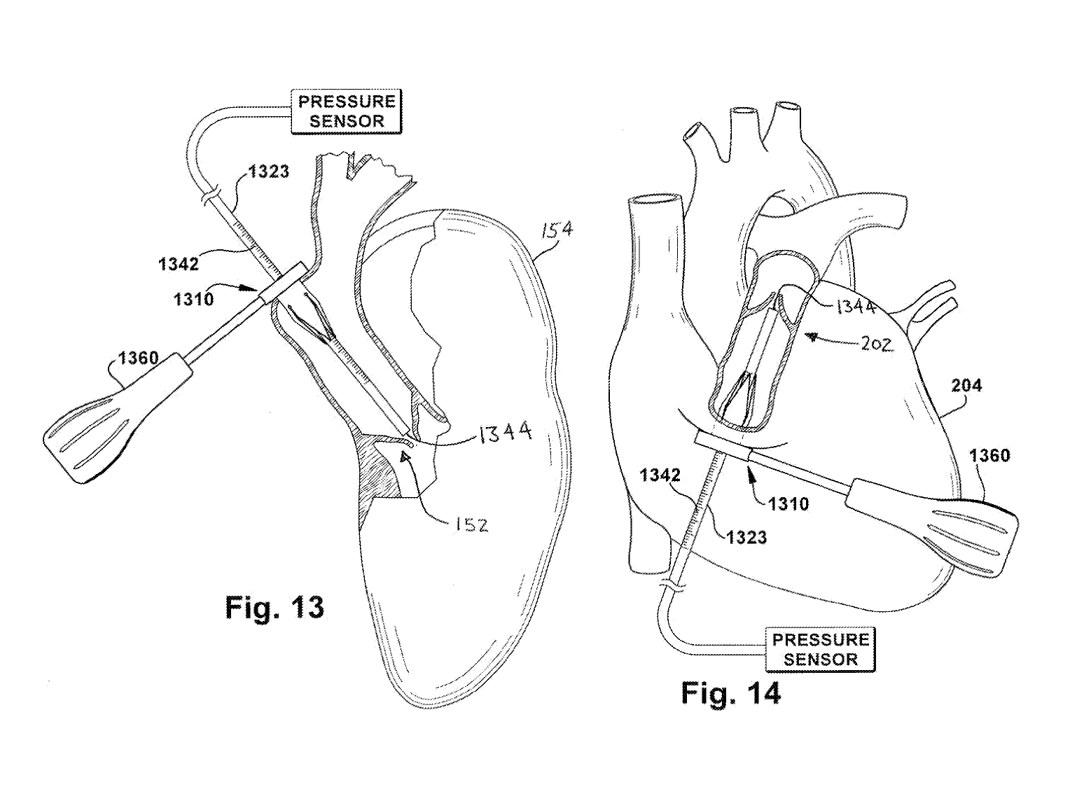

In FIGS. 13 and 14, an elongate sensor tube 1323 is depicted as being inserted into an introducer apparatus 1310 and into the heart 154, 204. The apparatus 1310 can be of the type shown and described herein with respect to FIGS. 8-12 or otherwise constructed consistent with one or more of the features described herein. Those skilled in the art will understand and appreciate that a flexible and/or curved tube 1323 may be utilized. In FIG. 13, the elongate tube 1323 is inserted at a position to provide a pressure measurement for implanting a heart valve prosthesis at a desired aortic position (e.g., at the aortic annulus 152). In FIG. 14, the elongate tube 1323 is inserted at a position to provide a pressure measurement for implanting a heart valve prosthesis at a desired pulmonic position (e.g., at the pulmonic valve 202).

A distal end of the elongate tube 1323 has been inserted through the introducer apparatus 1310 that so that respective members of the distal end portion are urged apart slightly by the elongate tube 1323 and thereby permit substantially free movement of the elongate tube through the introducer apparatus. The elongate tube 1323 further can include indicia 1342, such as corresponding to ruler markings, for providing an indication of measurement as to the distance which an end 1344 of the elongate tube has been inserted into the heart 154, 204. Accordingly, the measurement from the indicia 1342 enables a user to know the location of the pressure measurement. The introducer apparatus 1310 further includes a handle member 1360 for helping guide the introducer apparatus during the procedure.

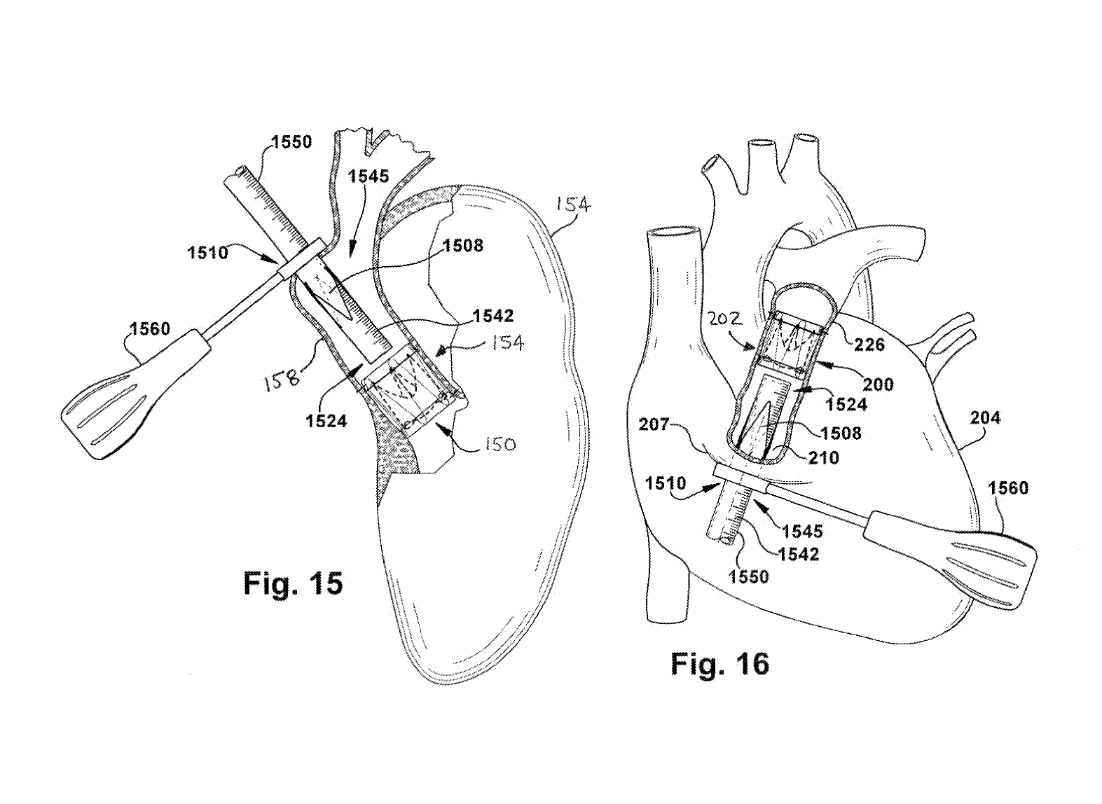

In FIGS. 15 and 16, a barrel 1550 of an implanter 1545 is depicted as being inserted into the introducer apparatus 1510 and into the heart 204 for two difference implantation positions. Specifically, FIG. 15 demonstrates an example that can be used for implanting a cardiac prosthesis 200 at the aortic position 152 and FIG. 16 demonstrates an example for implanting a cardiac prosthesis at the pulmonic position 202.

As shown in the examples of FIGS. 15 and 16, the barrel 1550 of the implanter 1545 is a substantially linear barrel, such as part of an implanter described herein (see, e.g., FIG. 3). Those skilled in the art will understand and appreciate that a flexible and/or curved barrel 1550 can also be utilized. The barrel 1550 is inserted at a position to provide a substantially linear or direct path for implanting the heart valve prosthesis 150, 200 at the desired position.

In FIGS. 15 and 16, an elongated distal portion 1524 of the barrel 1550, which is configured for carrying the prosthesis 200, has been inserted through the introducer apparatus 1510. The introducer apparatus 1510 is positioned and oriented at an angle to provide a substantially linear path (e.g., the central axis of the introducer apparatus is axially aligned with the implantation site). Thus, as the barrel is inserted through the introducer, respective jaw members of the distal end portion 1508 are urged apart to the open condition by the penetration of the barrel therethrough. The jaw members thus permit substantially free movement of the barrel through the introducer apparatus.

By inserting the introducer apparatus 1550 through the anterior wall 207 proximal the pulmonary artery 226, a substantially direct or generally linear implantation of the prosthesis 150, 200 can be performed with little or no cardiopulmonary bypass. As mentioned above, however, when the patient's defective valve is calcified, cardiopulmonary bypass will typically be performed for removing the calcified valve portions prior to implantation. The removing of the valve can be performed, for example, by employing a trocar or other tube inserted through the introducer apparatus 1510 or during an open heart procedure prior to implanting the prosthesis 150, 200. The introducer apparatus 1510 thus also facilitates such other procedures by providing a direct, linear path to the site where such procedures may be needed while also preventing backflow of blood through the introducer apparatus (e.g., by virtue of the structure—a valve or plug—located in the sidewall of the apparatus).

Those skilled in the art will understand and appreciate other possible paths through the heart or associated arteries or veins that could be employed for positioning the distal end 1524 of the barrel 1545 to enable implantation of the prosthesis 200 in accordance with an aspect of the present invention. The barrel 1545 further can include indicia 1542, such as corresponding to ruler markings, for providing an indication of measurement as to the distance which the end 1524 of the barrel has been inserted into the heart 204. Accordingly, the measurement from the indicia 1542 enables a user to discharge the prosthesis 200 at the desired implantation position; namely, into the aorta at the aortic position 154 shown in FIG. 15 and into the pulmonary artery 226 at the pulmonic position 202 shown in FIG. 16. Once at the desired position, the prosthesis 150, 200 can discharged or ejected from the barrel 1545: into the aorta 158, as illustrated in FIG. 15, and into the outflow of the right ventricle 210, as illustrated in FIG. 16. The introducer apparatus 1510 further includes a handle member 1360 for helping guide and stabilize the introducer apparatus during the procedure.

A system in accordance with one aspect of the present invention may comprise discharging means, guiding means, controlling means, and securing means. The discharging means may be the elongate member 40 or 1550, the implanter 100, 170, 222, or 1545, or other suitable equivalent. The guiding means may be the introducer apparatus 10, 102, 156, 206, 810, 910, 1010, 1110, 1210, 1310, or 1510, or other suitable equivalent. The controlling means may be the valve structure 920, 1121, or 1220, the plug member 1020, or other suitable equivalent. The securing means may be the handle member 860, 1360, or 1560, the threaded aperture 850, the threaded end 862 of the handle member 860, or other suitable equivalent.

The above system may further include plugging means, which may be the plug member 1020 or other suitable equivalent. The above system may still further include monitoring means, which may be the elongate tube 1023, 1123, or 1342, the central access port 1022, and/or the pressure sensor 1124, as well as suitable equivalent structures.

While the foregoing examples illustrate implanting heart valve prosthesis in the heart of a patient, those skilled in the art will understand and appreciate that the introducer/implanter combination can be utilized to implant other types of implantable cardiac articles, such as annuloplasty rings, stents, as well as other devices. Additionally, the barrel of an implanter or other elongate structure can be utilized as a trocar for performing surgical operations within the heart as may be required to facilitate adjustments of a heart valve prosthesis that has been discharged from an implanter or for adjusting the position of the heart valve prosthesis or other implanted device after its implantation. Additionally, those skilled in the art will understand and appreciate that the introducer apparatus and an implanter of sorts can be utilized for implanting other types of prosthesis and implantable devices associated with other anatomical structures or organs of the patient.

What has been described above includes examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

What is claimed is:

1. A substantially biocompatible introducer apparatus, comprising:
a flange;
a sleeve having a sidewall that extends longitudinally from the flange and terminates in a tapered distal end portion; and
a structure operatively associated with the sleeve and configured for substantially preventing flow of fluid in at least one axial direction from the distal end portion toward the flange.

2. The apparatus of claim 1, wherein the structure comprises a valve secured to an inner periphery of the sleeve proximal the flange.

3. The apparatus of claim 2, wherein the valve comprises a plurality of flap members resiliently engaging each other such that the plurality of flap members re-engage each other when an elongate member is withdrawn from a passage defined by an interior of the sidewall of the sleeve.

4. The apparatus of claim 3, wherein the plurality of flap members are molded to the inner periphery of the flange.

5. The apparatus of claim 1, wherein the structure comprises a plug member insertable at least partially into the sleeve at an opening defined by a radially inner periphery of the flange.

6. The apparatus of claim 5, wherein the plug member further comprises external threads; and
wherein the sleeve further comprises an internally threaded sidewall, the plug member being threaded into the internally threaded sidewall to prevent movement of fluid in the axial direction.

7. The apparatus of claim 6, wherein the plug member further comprises a central port for monitoring pressure at a distal side of the plug member.

8. The apparatus of claim 7, further comprising an elongate central access tube that extends longitudinally through the central port of the plug member and through the tapered distal end of the sleeve, the access tube include a mechanism that is positionable relative to the sleeve and configured to monitor pressure.

9. The apparatus of claim 1, further comprising an elongate central access tube that extends through the apparatus for monitoring pressure at a distal side of the structure.

10. The apparatus of claim 1, wherein the flange further comprises a radially extending, internally threaded aperture, the apparatus further comprising a handle threaded into the internally threaded aperture of the flange for facilitating insertion of the apparatus into a patient.

11. An implanter in combination with the apparatus of claim 1, the combination further comprising:
an implanter for discharging an article into a position within a patient, the implanter including an elongate member that terminates in a opening at a distal end thereof, at least a portion of the elongate member and the distal end being dimensioned and configured for axial passage through the sleeve of the apparatus.

12. An implantation system comprising:
an implanter including an elongate member that terminates in a opening at a distal end thereof, the implanter being configured for discharging an article through the opening and into a position within a patient; and
an introducer apparatus configured for guiding the implanter through an interior of the introducer apparatus to the position within the patient, the introducer apparatus including a flange, a sleeve having a sidewall that extends longitudinally from the flange and terminates in a distal end portion, and a structure operatively associated with the sleeve,
the structure at least partially located within an interior of the introducer apparatus for substantially preventing flow of fluid in at least a first axial direction.

13. The implantation system of claim 12, wherein the structure comprises a valve secured to an inner periphery of the sidewall, the valve comprising a plurality of flap members resiliently engaging each other such that the flap members re-engage each other when the elongate member is withdrawn from a passage defined by the sidewall of the introducer apparatus.

14. The implantation system of claim 12, wherein the structure comprises an externally threaded plug member engaging threads disposed at an inner periphery of the flange.

15. The implantation system of claim 12, wherein the structure comprises an externally threaded plug member having a central aperture extending therethrough the plug member, the implantation system further comprising a mechanism that is positionable within the central aperture for monitoring pressure near a distal side of the plug member.

16. The implantation system of claim 11, wherein the structure comprises jaw members extending axially from a generally cylindrical portion of the sidewall of the introducer apparatus, such that the jaw members define the distal end portion of the sleeve.

17. A system comprising:
means for discharging an article into a position within a patient;
means for guiding the discharging means to the position within the patient, the guiding means including a flange, a sleeve having a sidewall that extends longitudinally from the flange and terminates in a distal end portion, an elongate portion of the discharging means being insertable axially through at least a portion of the guiding means;
means for controlling axial flow of fluid through the guiding means; and
means for securing a handle member to the flange for facilitating insertion of the guiding means into the patient.

18. The system of claim 17, wherein the controlling means comprises a valve extending from an inner periphery of the sidewall of the sleeve proximal the flange, the valve including flap members resiliently engaging each other such that the flap members re-engage each other when the elongate portion of the discharging means is withdrawn from a passage defined by an interior of the sidewall of the sleeve.

19. The system of claim 17, wherein the controlling means prevents flow of fluid through the guiding means in at least one axial direction.

20. The system of claim 17, wherein the controlling means comprises means for plugging a passage defined by the sidewall of the sleeve, the plugging means disposed at an inner periphery of the flange in order to prevent movement of fluid in at least one axial direction.

21. The system of claim 17, further comprising means for monitoring pressure at a distal side of the guiding means.
